(19) United States
(12) Patent Application Publication
(10) Pub. No.: US 2025/0090758 A1
(43) Pub. Date: Mar. 20, 2025

(54) A BOTULINUM TOXIN SYRINGE

(71) Applicant: Thomas WEBB, Miranda (AU)

(72) Inventor: Thomas WEBB, Miranda (AU)

(21) Appl. No.: 18/968,584

(22) Filed: Dec. 4, 2024

Related U.S. Application Data
(63) Continuation of application No. PCT/AU2023/050490, filed on Jun. 6, 2023.

(30) Foreign Application Priority Data
Jun. 7, 2022 (AU) 2022901550

(51) Int. Cl. A61M 5/31; C12N 9/52

(52) U.S. Cl. CPC A61M 5/3129; C12N 9/52; A61M 2005/3126; C12Y 304/24069

(57) ABSTRACT

A botulinum toxin syringe having a barrel has volumetric marking graduations scaled at four units per 0.1 mL. These volumetric marking graduations allow for ease of unit conversion and can reduce the likelihood of overdosing or under dosing of botulinum toxin. The syringe may further comprise further volumetric marking graduations configured to be interchangeably used for administration of insulin or botulinum toxin. The syringe may further comprise further volumetric marking graduations configured for interchangeably use for administration of different types of botulinum toxin.

### FIELD OF THE INVENTION

This invention relates generally to a type of syringe for administration of botulinum toxin.

### BACKGROUND OF THE INVENTION

Insulin syringes are commonly used within the cosmetics industry for injection of botulinum toxin given their availability and sizing. One common type of insulin syringe is the BD ultra fine II™ needle syringe.

Insulin syringes comprise unit specific volumetric markings based on units of biologic activity.

The standard and most commonly used strength of insulin is U-100, which has 100 units of insulin per mL of fluid.

Insulin syringes are manufactured in 30-unit (0.3 mL) measures as shown in FIG. 1. However, 50-unit (0.5 mL) and 100-unit (1.0 ml) measures are also provided.

The size of the syringe needed depends on the required insulin dose wherein, for example, a 10 unit dose would be easier to administer using a 30 unit syringe as opposed to a 50 unit syringe.

Doses of all commercially available botulinum toxins are also expressed in terms of units of biologic activity. One unit of botulinum toxin corresponds to the calculated median intraperitoneal lethal dose (LD50) in female Swiss-Webster mice.

Insulin syringes are commonly used to administer botulinum toxin. However, unit volumetric scales of insulin and botulinum toxin are irreconcilable, necessitating mental conversion of insulin units to botulinum toxin units during administration of botulinum toxin using an insulin syringe.

For example, injection of two units of botulinum toxin nonintuitively requires the five unit graduation of the insulin syringe. Whereas the offset for smaller volumes can be more easily counted in sequence from the distal end of the barrel, it becomes difficult to mentally convert larger volumes wherein, for example, administering 10 units of botulinum toxin would nonintuitively require the 25 unit graduation on the insulin syringe.

Conversion of units between graduations becomes even more difficult. As shown in FIG. 1, the 30 unit insulin syringe has graduation increments of five making it quite difficult to administer nine units of botulinum toxin for example.

Errors in unit conversion can lead to under dosing or overdosing of botulinum toxin, with undesirous consequences.

The present invention seeks to provide a way which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a botulinum toxin syringe comprising a barrel having volumetric marking graduations scaled at four units per 0.1 mL, thereby avoiding the aforedescribed difficulties in conversion between units and significantly reducing the likelihood of overdosing or under dosing of botulinum toxin.

The syringe may further comprise further volumetric marking graduations configured for interchangeable use for administration of insulin or botulinum toxin.

In further embodiments, the syringe may further comprise further volumetric marking graduations configured for interchangeable use for administration of different types of botulinum toxin.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows an insulin syringe according to the prior art, such as the BD ultra fine II™ needle syringe; and

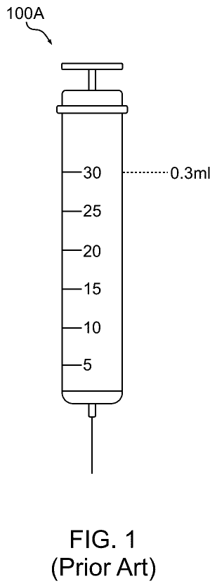

FIG. 2 shows a botulinum toxin syringe according to an embodiment of the present invention.

FIG. 3 shows a further embodiment wherein the botulinum toxin syringe comprises further volumetric markings having graduations on a different scale for use for botulinum toxin at a different dilution.

### DESCRIPTION OF EMBODIMENTS

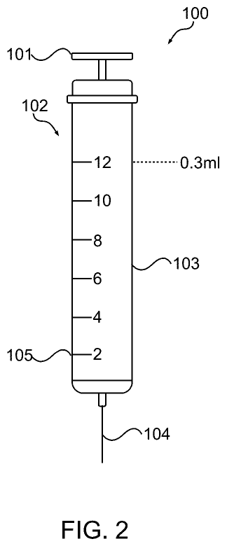

FIG. 2 shows a botulinum toxin syringe 100 comprising a transparent barrel 103 having a plunger 101 operative therein. A needle 104 extends from a distal end of the barrel. The needle 104 is preferably a 32G 8 mm needle.

The barrel 103 comprises volumetric markings 102.

The volumetric markings 102 comprise graduations 105 scaled at four units per 0.1 mL. For example, 12 units would correspond with approximately 0.3 mL as shown.

In embodiments, a larger syringe 100 may be provided wherein the maximum volumetric marking graduation is 20 which corresponds with 0.5 mL. In further embodiments, an even larger syringe 100 may be provided wherein the maximum volumetric marking graduation is 40 which corresponds with 1 mL.

The graduations 105 may increment in twos. In the embodiment shown, the volumetric marking graduations 105 increment in twos and do not include any other graduations therebetween.

The maximum graduation 105 may be between 8 and 16, thereby conferring a suitably sized measure for typical doses of botulinum toxin. The maximum graduation 105 may be 12 according to the embodiment shown in FIG. 2.

In use, the syringe 100 may be used to inject a number of units of botulinum toxin according to the graduations 105.

In embodiments, the syringe 100 may comprise further volumetric markings 102 scaled at 10 units per 0.1 mL for insulin units so that the syringe 100 may be interchangeably used for insulin or botulinum toxin.

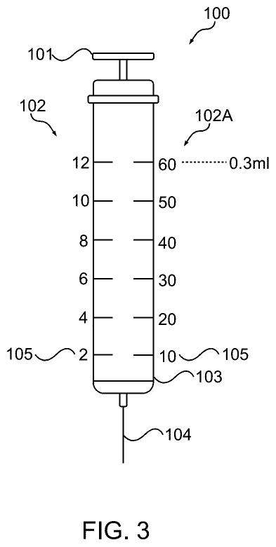

According to the embodiment shown in FIG. 2, the volumetric markings 102 are designed for botulinum toxin at a certain dilution. However, FIG. 3 shows a further embodiment wherein the botulinum toxin syringe 100 comprises further volumetric markings 102A having graduations 105 on a different scale for use for botulinum toxin at a different dilution.

Preferably, the further volumetric markings 102 has graduations 105 scaled at 20 units to 0.1 mL.

Specifically, the further volumetric markings are scaled at unit increments of 10 and have graduations 105 from 10 to 60, the 60 unit graduation 105 corresponding with a 0.3 mL volume.

Preferably the graduations 105 of both volumetric markings 102, 102A align. For example, the two unit graduation 105 of volumetric markings 102 aligns with the 10 unit graduation 105 of volumetric markings 102A and so on.

As such, the syringe of FIG. 3 allows for convenient use for a botulinum toxin having a 12 unit concentration at 0.3 mL or another botulinum toxin having a 60 unit concentration at 0.3 mL. The appropriate volumetric markings 102 or 102A would be selected for use depending on the type of botulinum toxin used with the syringe.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

1. A botulinum toxin syringe comprising a barrel having volumetric marking graduations scaled at 4 units per 0.1 mL.

2. The syringe as claimed in claim 1, wherein the volumetric marking graduations increment in twos.

3. The syringe as claimed in claim 1, wherein the volumetric marking graduations increment in twos and do not include any other graduations therebetween.

4. The syringe as claimed in claim 1, wherein the maximum volumetric marking graduation is between 8 and 16.

5. The syringe as claimed in claim 4, wherein the maximum volumetric marking graduation is 12 which corresponds with 0.3 mL.

6. The syringe as claimed in claim 1, wherein the maximum volumetric marking graduation is 20 which corresponds with 0.5 mL.

7. The syringe as claimed in claim 1, wherein the maximum volumetric marking graduation is 40 which corresponds with 1 mL.

8. The syringe as claimed in claim 1, further comprising further volumetric markings are scaled at 10 units per 0.1 mL.

9. The syringe as claimed in claim 1, further comprising further volumetric markings having graduations on a different scale.

10. The syringe as claimed in claim 9, wherein the further volumetric markings are scaled at 20 units to 0.1 mL.

11. The syringe as claimed in claim 10, wherein the further volumetric markings are scaled at unit increments of 10.

12. The syringe as claimed in claim 11, wherein the further volumetric markings have graduations from 10 to 60.

13. The syringe as claimed in claim 12, wherein the further volumetric markings have a 60 unit graduation corresponding with a 0.3 mL.

14. The syringe as claimed in claim 9, wherein graduations of the volumetric markings and the further volumetric markings align.

15. The syringe as claimed in claim 14, wherein the two unit graduation of volumetric markings aligns with the 10 unit graduation of the volumetric markings.

16. A method of injecting botulinum toxin using the syringe as claimed in claim 1, the method comprising injecting a number of units of botulinum toxin according to the graduations, the graduations scaled at 4 units to 0.1 mL.

17. A method of injecting botulinum toxin or insulin using the syringe as claimed in claim 8, the method comprising selecting the volumetric markings or the further volumetric markings for use depending on whether botulinum toxin or insulin is being administered.

18. A method of injecting botulinum toxin using the syringe as claimed in claim 9, the method comprising selecting the volumetric markings or the further volumetric markings for use depending on a type of botulinum toxin being administered using the syringe.

19. The method as claimed in claim 18, wherein the graduations of the volumetric markings are scaled at 4 units to 0.1 mL and the graduations of the further volumetric markings are scaled at 20 units to 0.1 mL.
